(19) United States
(12) Patent Application Publication
(10) Pub. No.: US 2010/0256083 A1
(43) Pub. Date: Oct. 7, 2010

(54) ORGAN PROTECTION, PRESERVATION AND RECOVERY

(75) Inventor: Geoffrey P. Dobson, Queensland (AU)

Correspondence Address: VENABLE LLP, P.O. BOX 34385, WASHINGTON, DC, 20043-9998, US

(73) Assignee: HIBERNATION THERAPEUTICS LIMITED, QUEENSLAND (AU)

(21) Appl. No.: 12/670,452

(22) PCT Filed: Jul. 25, 2008
(86) PCT No.: PCT/AU08/01086

§ 371 Date: Jun. 17, 2010

(30) Foreign Application Priority Data
Jul. 25, 2007 (AU) 2007903999

(51) Int. Cl. A61K 31/7076; A61P 9/00

(52) U.S. Cl. 514/46

(57) ABSTRACT

This application describes compositions, methods of treatment, and methods of manufacturing a medicament for reducing injury or damage to cells, tissues or organs during ischemia, reperfusion, or following ischemia or trauma. The methods for reducing damage to a cell, tissue or organ comprise administering an effective amount of a composition including (i) a potassium channel opener or agonist and/or adenosine receptor agonist; and (ii) an antiarrhythmic agent. The methods may further include postconditioning the cell, tissue or organ.

### FIELD OF THE INVENTION

This invention relates to methods of reducing injury to cells, tissues or organs during ischemia or reperfusion. The invention also relates to reducing damage to cells, tissues or organs that may result from ischemia or some form of injury or trauma.

### BACKGROUND OF THE INVENTION

Ischaemic heart disease remains the leading cause of death and morbidity in Australia and other industrialised nations. A large percentage of deaths are due to ventricular fibrillation (VF) secondary to metabolic, ionic and functional disturbances after the onset of ischaemia. Restoration of coronary flow within 15 min can lead to full recovery, but it can also predispose the myocardium to potentially fatal arrhythmias and myocardial stunning during recovery. If ischaemia persists beyond the ‘reversible’ window, the heart will undergo progressive loss of ATP and cell death from necrosis and apoptosis.

Over the past decade, considerable research has focused on pharmacological strategies to prevent, delay or attenuate ischaemia-reperfusion injury by targeting cell receptors (e.g., adenosine A1 and A3, opioid and adrenergic), ion channels (e.g., Na⁺ fast, sarcolemmal K_{ATP} and mitochondrial K_{ATP}, Cl⁻, Ca²⁺), exchangers (e.g., Na⁺/H⁺, Na⁺/Ca²⁺) and intracellular signalling pathways (e.g., protein kinase C, tyrosine protein kinase, guanylate cyclase) from ischaemia-reperfusion injury. WO00/56145, WO 2004/056180, and WO 2004/056181, for example, describe pharmacological strategies useful to reduce damage to a cell tissue or organ during reperfusion or ischemia.

The present invention is directed toward overcoming or at least alleviating one or more of the difficulties and deficiencies of the prior art.

### SUMMARY OF THE INVENTION

This invention is directed to improved methods of reducing injury or damage to cells, tissues or organs during ischemia or reperfusion.

In one embodiment, the invention provides a method for reducing damage to a cell, tissue or organ following ischemia comprising: administering an effective amount of a composition including (i) a potassium channel opener or agonist and/or adenosine receptor agonist; and (ii) an antiarrhythmic agent; and postconditioning the cell, tissue or organ.

In another embodiment, the present invention provides a method for reducing damage to a cell, tissue or organ following trauma comprising: administering an effective amount of a composition including (i) a potassium channel opener or agonist and/or adenosine receptor agonist; and (ii) an antiarrhythmic agent; and postconditioning the cell, tissue or organ.

In a further embodiment, the present invention provides a method for reducing damage to a cell, tissue or organ prior to or during ischemia or reperfusion: comprising administering an effective amount of a composition including (i) a potassium channel opener or agonist and/or adenosine receptor agonist; and (ii) an antiarrhythmic agent; and postconditioning the cell, tissue or organ.

In another embodiment the invention provides a method for reducing damage to a cell, tissue or organ following ischemia comprising: administering an effective amount of a composition including (i) a potassium channel opener or agonist and/or adenosine receptor agonist; and (ii) an antiarrhythmic agent and (iii) an opioid. In this embodiment, the method may further include the step of post-conditioning the cell, tissue or organ.

In another embodiment, the present invention provides a method for reducing damage to a cell, tissue or organ following trauma comprising: administering an effective amount of a composition including (i) a potassium channel opener or agonist and/or adenosine receptor agonist; (ii) an antiarrhythmic agent; and (iii) an opioid. According to this embodiment, the method may further include the step of post-conditioning the cell, tissue or organ.

In a further embodiment, the present invention provides a method for reducing damage to a cell, tissue or organ prior to or throughout ischemia or reperfusion: administering an effective amount of a composition including (i) a potassium channel opener or agonist and/or adenosine receptor agonist; and (ii) an antiarrhythmic agent and (iii) an opioid. According to this embodiment, the method may further include the step of post-conditioning the cell, tissue or organ.

The composition may be administered to the cell, tissue or organ respectively. Also, it may be administered to a patient, as described below.

In yet another embodiment, there is provided a method for reducing injury to a cell, tissue or organ including:
providing in a suitable container a composition as described herein;
providing one or more nutrient molecules selected from the group consisting of blood, blood products, artificial blood and a source of oxygen;
optionally aerating the composition with the oxygen (for example, in the case of isolated organs) or combining the nutrient molecules with the composition, or both; and
placing the tissue, cell or organ in contact with the combined composition under conditions sufficient to reduce injury.

The methods of the invention are applicable to any cell, tissue or organ. Examples include where the cell is a myocyte, endothelial cell, smooth-muscle cell, neutrophil, platelet and other inflammatory cells, or the tissue is heart tissue, or the organ is a heart.

The compositions administered may also include additional components selected from one or more of potassium channel openers or agonists, adenosine receptor agonist, opioid, at least one compound for reducing uptake of water, sodium/hydrogen exchange inhibitor, antioxidant, calcium channel blocker and a source of magnesium in an amount for increasing the amount of magnesium in a cell in body tissue.

In another aspect of the invention there is provided a composition for reducing damage to a cell, tissue or organ during ischemia or reperfusion or following ischemia or trauma comprising:
a potassium channel opener or agonist and/or adenosine receptor agonist;
an antiarrhythmic agent; and
an opioid.

In a further aspect of the invention there is provided use of a composition as described herein for the preparation of a medicament for reducing damage to a cell, tissue or organ during ischemia, or reperfusion, or following ischemia or trauma.

### DETAILED DESCRIPTION OF THE INVENTION

This invention is directed to improved methods of reducing injury or damage to cells, tissues or organs during ischemia or reperfusion. The invention also has application to reducing damage to cells, tissues or organs that may result from ischemia or some form of injury or trauma.

In one form, the invention provides a method for reducing damage to a cell, tissue or organ following ischemia or trauma or prior to or during ischemia or reperfusion comprising: administering an effective amount of a composition including (i) a potassium channel opener or agonist and/or adenosine receptor agonist; and (ii) an antiarrhythmic agent; and postconditioning the cell, tissue or organ.

The inventor has found that the administration of a composition including (i) a potassium channel opener or agonist and/or adenosine receptor agonist; and (ii) an antiarrhythmic agent prior to or during ischemia or reperfusion together with postconditioning the cell, tissue or organ reduces cell damage resulting from ischemia or reperfusion.

The methods according to the invention have wide clinical significance in protecting the human heart from ischaemia-reperfusion injury during heart surgery (on-pump and off-pump), coronary interventions (balloon and stent), acute ischaemic syndromes, arrhythmia management and organ transplantation. For example, the methods offer surgeons who employ local preconditioning therapy during off-pump heart surgery with a safer alternative. It may also find utility in assisting cardiologists to reduce arrhythmias and ischaemia-reperfusion injury during angioplasty/stent interventions.

“Postconditioning” is a series of rapid intermittent mechanical interruptions of blood flow in the early phase of reperfusion (this is described in PCT patent application WO 2006/069170 in the name of Emory University). Postconditioning may also be elicited pharmacologically using drugs or enhancers to activate the receptors and chemical and biochemical pathways believed to be associated with postconditioning. Postconditioning may be applicable in the “off-pump” and “on-pump” surgery as well as angioplasty because the reperfusion can be controlled by the surgeon or the interventionist. Postconditioning during angioplasty has been shown to be effective in reducing infarct size by 30%, even as far as 7 days from the procedure.

Unlike preconditioning, which requires foreknowledge of the ischemic event, postconditioning can be applied at the onset of the medical treatment, for example angioplasty, cardiac surgery and transplantation. The methods according to the invention may also be useful for treating patients that have experienced a trauma that may have resulted from injury in the battlefield or accident.

“Injury” can be broadly characterised as reversible and irreversible cell injury. For example, reversible cell injury can lead to heart dysfunction usually from arrhythmias and/or stunning. Stunning is normally characterised as loss of left pump function during restoration of blood flow following periods of ischemia. If severe, it can lead to the death of the heart, usually from arrhythmias, even though the heart cells themselves are not initially dead. Irreversible injury by definition arises from actual cell death which may be fatal depending upon the extent of the injury. The amount of cell death can be measured as infarct size. During recovery from cardioplegic arrest, if the conditions are adequate, the heart can be restored substantially to normal function of the tissue by reperfusion, with minimal infarct size. The most common ways to assess return of function of a heart are by measuring pressures that the heart can generate; heart pump flow; and the electrical activity of the heart. This data is then compared to data measured from pre-arrest conditions. In this specification the terms “injury” and “damage” may be used interchangeably.

The term “tissue” is used herein in its broadest sense and refers to any part of the body exercising a specific function including organs and cells or parts thereof, for example, cell lines or organelle preparations. Other examples include conduit vessels such as arteries or veins or circulatory organs such as the heart, respiratory organs such as the lungs, urinary organs such as the kidneys or bladder, digestive organs such as the stomach, liver, pancreas or spleen, reproductive organs such as the scrotum, testis, ovaries or uterus, neurological organs such as the brain, germ cells such as spermatozoa or ovum and somatic cells such as skin cells, heart cells (ie, myocytes), nerve cells, brain cells or kidney cells.

The term “organ” is used herein in its broadest sense and refers to any part of the body exercising a specific function including tissues and cells or parts thereof, for example, endothelium, epithelium, blood brain barrier, cell lines or organelle preparations. Other examples include circulatory organs such as the blood vessels, heart, respiratory organs such as the lungs, urinary organs such as the kidneys or bladder, digestive organs such as the stomach, liver, pancreas or spleen, reproductive organs such as the scrotum, testis, ovaries or uterus, neurological organs such as the brain, germ cells such as spermatozoa or ovum and somatic cells such as skin cells, heart cells i.e., myocytes, nerve cells, brain cells or kidney cells.

The body may be a human or an animal such as a livestock animal (eg, sheep, cow or horse), laboratory test animal (eg, mouse, rabbit or guinea pig) or a companion animal (eg, dog or cat), particularly an animal of economic importance. Preferably, the body is human.

It will also be understood that the term “comprises” (or its grammatical variants) as used in this specification is equivalent to the term “includes” and should not be taken as excluding the presence of other elements or features.

The invention described in this specification largely relates to compositions, methods of treatment, and methods of manufacturing a medicament for treatment involving a composition which is described as containing these components (i) and (ii) (and additional components where applicable). For convenience, this composition will be referred to in this specification as the “composition” or “composition useful in methods according to the invention”, although there are a number of combinations of components embodying the invention which are compositions useful in the invention. Moreover, as explained particularly in WO00/56145, the components (i) and (ii) may be present in a concentration which arrests, or does not arrest a heart referred to as an “arresting” concentration of the composition and a “non-arresting” concentration of the composition. In one form, the arresting composition contains adenosine and lignocaine, each at greater than 0.1 mM (and preferably below 20 mM). The arresting composition may in some circumstances be referred to as a “cardioplegia solution”. In one form of the non-arresting composition, adenosine and lignocaine are both below 0.1 mM and preferably 50 nM to 95 μM, or more preferably from 1 μM to 90 μM.

If potassium is present in the composition it will typically be present at physiological levels. This means that when the composition is administered, the cell membrane remains in a more physiological polarised state thereby minimising potential damage to the cell, tissue or organ. High concentrations or concentrations above physiological levels of potassium would result in a hyperkalemic composition. At these concentrations the heart would be arrested alone from the depolarisation of the cell membrane.

One advantage of using physiological concentrations of potassium is that it renders the present composition less injurious to the subject, in particular paediatric subjects such as neonates/infants. High potassium has been linked to an accumulation of calcium which may be associated with irregular heart beats during recovery, heart damage and cell swelling. Neonates/infants are even more susceptible than adults to high potassium damage during cardiac arrest. After surgery a neonate/infant's heart may not return to normal for many days, sometimes requiring intensive therapy or life support.

In the embodiments of the invention described above and below, component (i) of the composition may be an adenosine receptor agonist. While this obviously includes adenosine itself, the “adenosine receptor agonist” may be replaced or supplemented by a compound that has the effect of raising endogenous adenosine levels. This may be particularly desirable where the compound raises endogenous adenosine levels in a local environment within a body. The effect of raising endogenous adenosine may be achieved by a compound that inhibits cellular transport of adenosine and therefore removal from circulation or otherwise slows its metabolism and effectively extends its half-life (for example, dipyridamole) and/or a compound that stimulates endogenous adenosine production such as purine nucleoside analogue Acadesine™ or AICA-riboside (5-amino-4-imidazole carboxamide ribonucleoside). Acadesine is also a competitive inhibitor of adenosine deaminase (Ki=362 μM in calf intestinal mucosa.) Acadesine™ is desirably administered to produce a plasma concentration of around 50 μM but may range from 1 μM to 1 mM or more preferably from 20 to 200 μM. Acadesine™ has shown to be safe in humans from doses given orally and/or intravenous administration at 10, 25, 50, and 100 mg/kg body weight doses.

In addition to the adenosine receptor agonist, or instead of the adenosine receptor agonist, component (i) of the composition may be a potassium channel opener.

Potassium channel openers are agents which act on potassium channels to open them through a gating mechanism. This results in efflux of potassium across the membrane along its electrochemical gradient which is usually from inside to outside of the cell. Thus potassium channels are targets for the actions of transmitters, hormones, or drugs that modulate cellular function. It will be appreciated that the potassium channel openers include the potassium channel agonists which also stimulate the activity of the potassium channel with the same result. It will also be appreciated that there are diverse classes of compounds which open or modulate different potassium channels; for example, some channels are voltage dependent, some rectifier potassium channels are sensitive to ATP depletion, adenosine and opioids, others are activated by fatty acids, and other channels are modulated by ions such as sodium and calcium (ie. channels which respond to changes in cellular sodium and calcium). More recently, two pore potassium channels have been discovered and thought to function as background channels involved in the modulation of the resting membrane potential.

Potassium channel openers may be selected from the group consisting of: nicorandil, diazoxide, minoxidil, pinacidil, aprikalim, cromokulim and derivative U-89232, P-1075 (a selective plasma membrane KATP channel opener), emakalim, YM-934, (+)-7,8-dihydro-6,6-dimethyl-7-hydroxy-8-(2-oxo-1-piperidinyl)-6H-pyrano[2,3-f]benz-2,1,3-oxadiazole (NIP-121), RO316930, RWJ29009, SDZPCO400, rimakalim, symakalim, YM099, 2-(7,8-dihydro-6,6-dimethyl-6H-[1,4]oxazino[2,3-f][2,1,3]benzoxadiazol-8-yl) pyridine N-oxide, 9-(3-cyanophenyl)-3,4,6,7,9,10-hexahydro-1,8-(2H,5H)-acridinedione (ZM244085), [(9R)-9-(4-fluoro-3-125iodophenyl)-2,3,5,9-tetrahydro-4H-pyrano[3,4-b]thieno[2,3-e]pyridin-8(7H)-one-1,1-dioxide] ([125I]A-312110), (−)—N-(2-ethoxyphenyl)-N′-(1,2,3-trimethylpropyl)-2-nitroethene-1,1-diamine (Bay X 9228), N-(4-benzoyl phenyl)-3,3,3-trifluoro-2-hydroxy-2-methylpropionamine (ZD6169), ZD6169 (KATP opener) and ZD0947 (KATP opener), WAY-133537 and a novel dihydropyridine potassium channel opener, A-278637. In addition, potassium channel openers may be selected from BK-activators (also called BK-openers or BK(Ca)-type potassium channel openers or large-conductance calcium-activated potassium channel openers) such as benzimidazolone derivatives NS004 (5-trifluoromethyl-1-(5-chloro-2-hydroxyphenyl)-1,3-dihydro-2H-benzimidazole-2-one), NS1619 (1,3-dihydro-1-[2-hydroxy-5-(trifluoromethyl)phenyl]-5-(trifluoromethyl)-2H-benzimidazol-2-one), NS1608 (N-(3-(trifluoromethyl)phenyl)-N′-(2-hydroxy-5-chlorophenyl)urea), BMS-204352, retigabine (also GABA agonist). There are also intermediate (eg. benzoxazoles, chlorzoxazone and zoxazolamine) and small-conductance calcium-activated potassium channel openers.

In addition, potassium channel openers may act as indirect calcium antagonists, ie they act to reduce calcium entry into the cell by shortening the cardiac action potential duration through the acceleration of phase 3 repolarisation, and thus shorten the plateau phase. Reduced calcium entry is thought to involve L-type calcium channels, but other calcium channels may also be involved.

Some embodiments of the invention utilise direct calcium antagonists, the principal action of which is to reduce calcium entry into the cell. These are selected from at least five major classes of calcium channel blockers as explained in more detail below. It will be appreciated that these calcium antagonists share some effects with potassium channel openers, particularly ATP-sensitive potassium channel openers, by inhibiting calcium entry into the cell.

Adenosine as well as functioning as an adenosine receptor agonist is also particularly preferred as the potassium channel opener or agonist. Adenosine is capable of opening the potassium channel, hyperpolarising the cell, depressing metabolic function, possibly protecting endothelial cells, enhancing preconditioning of tissue and protecting from ischaemia or damage. Adenosine is also an indirect calcium antagonist, vasodilator, antiarrhythmic, antiadrenergic, free radical scavenger, arresting agent, anti-inflammatory agent (attenuates neutrophil activation), analgesic, metabolic agent and possible nitric oxide donor. More recently, adenosine is known to inhibit several steps which can lead to slowing the blood clotting process. In addition, elevated levels of adenosine in the brain has been shown to cause sleep and may be involved in different forms or dormancy. An adenosine analogue, 2-chloro-adenosine, may be used.

Suitable adenosine receptor agonists may be selected from: N⁶-cyclopentyladenosine (CPA), N-ethylcarboxamido adenosine (NECA), 2-[p-(2-carboxyethyl)phenethyl-amino-5′-N-ethylcarboxamido adenosine (CGS-21680), 2-chloroadenosine, N⁶-[2-(3,5-demethoxyphenyl)-2-(2-methoxyphenyl]ethyladenosine, 2-chloro-N⁶-cyclopentyladenosine (CCPA), N-(4-aminobenzyl)-9-[5-(methylcarbonyl)-beta-D-robofuranosyl]-adenine (AB-MECA), ([IS-[1a,2b,3b,4a(S*)]]-4-7-[[2-(3-chloro-2-thienyl)-1-methyl-propyl]amino]-3H-imidazole[4,5-b]pyridyl-3-yl]cyclopentane carboxamide (AMP579), N⁶-(R)-phenylisopropyladenosine (R-PLA), aminophenylethyladenosine (APNEA) and_cyclohexyladenosine (CHA). Others include full adenosine A1 receptor agonists such as N-[3-(R)-tetrahydrofuranyl]-6-aminopurine riboside (CVT-510), or partial agonists such as CVT-2759 and allosteric enhancers such as PD81723. Other agonists include N6-cyclopentyl-2-(3-phenylaminocarbonyltriazene-1-yl)adenosine (TCPA), a very selective agonist with high affinity for the human adenosine A1 receptor, and allosteric enhancers of A1 adenosine receptor includes the 2-amino-3-naphthoylthiophenes. Preferably, the A1 adenosine receptor agonist is CCPA.

It will be appreciated that anti-adrenergics such as beta-blockers, for example, esmolol, atenolol, metoprolol and propranolol could be used instead of or in combination with the potassium channel opener to reduce calcium entry into the cell. Preferably, the beta-blocker is esmolol. Similarly, alpha(1)-adrenoceptor-antagonists such as prazosin, could be used instead of or in combination with the potassium channel opener to reduce calcium entry into the cell and therefore calcium loading. Preferably, the antiadrenergic is a beta-blocker. Preferably the beta-blocker is esmolol.

Adenosine is also known to indirectly inhibit the sodium-calcium exchanger which would reduce cell sodium and calcium loading. It will be appreciated that inhibitors of the sodium-calcium exchanger would lead to reduced calcium entry and magnify the effect of adenosine. Na⁺/Ca²⁺exchange inhibitors may include benzamyl, KB-R7943 (2-[4-(4-Nitrobenzyloxy)phenyl]ethyl]isothiourea mesylate) or SEA0400 (2-[4-[(2,5-difluorophenyl)methoxy]phenoxy]-5-ethoxyaniline).

Some embodiments of the invention utilise direct calcium antagonists, the principal action of which is to reduce calcium entry into the cell. Such compounds may be selected from calcium channel blockers from three different classes: 1,4-dihydropyridines (eg. nitrendipine), phenylalkylamines (eg. verapamil), and the benzothiazepines (e.g. diltiazem, nifedipine). It will be appreciated that these calcium antagonists share some effects with potassium channel openers, particularly ATP-sensitive potassium channel openers, by inhibiting calcium entry into the cell.

Calcium channel blockers are also called calcium antagonists or calcium blockers. They are often used clinically to decrease heart rate and contractility and relax blood vessels. They may be used to treat high blood pressure, angina or discomfort caused by ischaemia and some arrhythmias, and they share many effects with beta-blockers (see discussion above).

Five major classes of calcium channel blockers are known with diverse chemical structures: 1. Benzothiazepines: eg Diltiazem, 2. Dihydropyridines: eg nifedipine, Nicardipine, nimodipine and many others, 3. Phenylalkylamines: eg Verapamil, 4. Diarylaminopropylamine ethers: eg Bepridil, 5. Benzimidazole-substituted tetralines: eg Mibefradil.

The traditional calcium channel blockers bind to L-type calcium channels (“slow channels”) which are abundant in cardiac and smooth muscle which helps explain why these drugs have selective effects on the cardiovascular system. Different classes of L-type calcium channel blockers bind to different sites on the alpha1-subunit, the major channel-forming subunit (alpha2, beta, gamma, delta subunits are also present). Different sub-classes of L-type channel are present which may contribute to tissue selectivity. More recently, novel calcium channel blockers with different specificities have also been developed for example, Bepridil, is a drug with Na+ and K+ channel blocking activities in addition to L-type calcium channel blocking activities. Another example is Mibefradil, which has T-type calcium channel blocking activity as well as L-type calcium channel blocking activity.

Three common calcium channel blockers are diltiazem (Cardizem), verapamil (Calan) and Nifedipine (Procardia). Nifedipine and related dihydropyridines do not have significant direct effects on the atrioventricular conduction system or sinoatrial node at normal doses, and therefore do not have direct effects on conduction or automaticity. While other calcium channel blockers do have negative chronotropic/dromotropic effects (pacemaker activity/conduction velocity). For example, Verapamil (and to a lesser extent diltiazem) decreases the rate of recovery of the slow channel in AV conduction system and SA node, and therefore act directly to depress SA node pacemaker activity and slow conduction. These two drugs are frequency- and voltage-dependent, making them more effective in cells that are rapidly depolarizing. Verapamil is also contraindicated in combination with beta-blockers due to the possibility of AV block or severe depression of ventricular function. In addition, mibefradil has negative chronotropic and dromotropic effects. Calcium channel blockers (especially verapamil) may also be particularly effective in treating unstable angina if underlying mechanism involves vasospasm.

Omega conotoxin MVIIA (SNX-111) is an N type calcium channel blocker and is reported to be 100-1000 fold more potent than morphine as an analgesic but is not addictive. This conotoxin is being investigated to treat intractible pain. SNX-482 a further toxin from the venom of a carnivorous spider venom, blocks R-type calcium channels. The compound is isolated from the venom of the African tarantula, Hysterocrates gigas, and is the first R-type calcium channel blocker described. The R-type calcium channel is believed to play a role in the body's natural communication network where it contributes to the regulation of brain function. Other Calcium channel blockers from animal kingdom include Kurtoxin from South African Scorpion, SNX-482 from African Tarantula, Taicatoxin from the Australian Taipan snake, Agatoxin from the Funnel Web Spider, Atracotoxin from the Blue Mountains Funnel Web Spider, Conotoxin from the Marine Snail, HWTX-I from the Chinese bird spider, Grammotoxin SIA from the South American Rose Tarantula. This list also includes derivatives of these toxins that have a calcium antagonistic effect.

Direct ATP-sensitive potassium channel openers (eg nicorandil, aprikalem) or indirect ATP-sensitive potassium channel openers (eg adenosine, opioids) are also indirect calcium antagonists and reduce calcium entry into the tissue. One mechanism believed for ATP-sensitive potassium channel openers also acting as calcium antagonists is shortening of the cardiac action potential duration by accelerating phase 3 repolarisation and thus shortening the plateau phase. During the plateau phase the net influx of calcium may be balanced by the efflux of potassium through potassium channels. The enhanced phase 3 repolarisation may inhibit calcium entry into the cell by blocking or inhibiting L-type calcium channels and prevent calcium (and sodium) overload in the tissue cell.

Calcium channel blockers can be selected from nifedipine, nicardipine, nimodipine, nisoldipine, lercanidipine, telodipine, angizem, altiazem, bepridil, amlodipine, felodipine, isradipine and cavero and other racemic variations. In addition, it will be appreciated that calcium entry could be inhibited by other calcium blockers which could be used instead of or in combination with adenosine and include a number of venoms from marine or terrestrial animals such as the omega-conotoxin GVIA (from the snail conus geographus) which selectively blocks the N-type calcium channel or omega-agatoxin IIIA and IVA from the funnel web spider Agelelnopsis aperta which selectively blocks R- and P/Q-type calcium channels respectively. There are also mixed voltage-gated calcium and sodium channel blockers such as NS-7 to reduce calcium and sodium entry and thereby assist cardioprotection. Preferably the calcium channel blocker is nifedipine.

In a preferred form, the potassium channel opener or agonist and/or an adenosine receptor agonist has a blood half-life of less than one minute, preferably less than 20 seconds.

The composition useful in methods according to the invention also includes an antiarrhythmic agent. Antiarrhythmic agents are a group of pharmaceuticals that are used to suppress fast rhythms of the heart (cardiac arrhythmias). The following table indicates the classification of these agents.

| | | Repolarisation | |
|---|---|---|---|
| CLASS | Channel effects | Time | Drug Examples |
| IA | Sodium block | Prolongs | Quinidine, |
| | | | disopyramide, |
| | | | Procaine |
| IB | Sodium block | Shortens | Lidocaine, |
| | | | phenytoin, |
| | | | mexiletine, |
| | | | Tocainide |
| IC | Sodium block | Unchanged | Flecainide |
| | | | Propafenone, |
| | | | moricizine |
| II | Phase IV | Unchanged | Beta-blockers |
| | (depolarising | | including sotalol |
| | current); Calcium | | |
| | channel | | |
| III | Repolarising | Markedly prolongs | Amiodarone, |
| | Potassium | | Sotalol, bretylium |
| | Currents | | |
| IVA | AV nodal calcium | Unchanged | Verapamil, |
| | block | | diltiazem |
| IVB | Potassium | Unchanged | Adenosine, ATP |
| | channel openers | | |

It will also be appreciated that the antiarrhythmic agent may induce local anaesthesia (or otherwise be a local anaesthetic), for example, mexiletine, diphenylhydantoin, prilocalne, procaine, mepivocaine, quinidine, disopyramide and Class 1B antiarrhythmic agents.

Preferably, the antiarrhythmic agent is a class I or class III agent. Amiodarone is a preferred Class III antiarrhythmic agent. More preferably, the antiarrhythmic agent blocks sodium channels. More preferably, the antiarrhythmic agent is a class IB antiarrhythmic agent. Class 1B antiarrhythmic agents include lignocaine or derivatives thereof, for example, QX-314.

Preferably the class 1B antiarrhythmic agent is Lignocaine. In this specification, the terms “lidocaine” and “lignocaine” are used interchangeably. Lignocaine is also known to be capable of acting as a local anaesthetic probably by blocking sodium fast channels, depressing metabolic function, lowering free cytosolic calcium, protecting against enzyme release from cells, possibly protecting endothelial cells and protecting against myofilament damage. At lower therapeutic concentrations lignocaine normally has little effect on atrial tissue, and therefore is ineffective in treating atrial fibrillation, atrial flutter, and supraventricular tachycardias. Lignocaine is also a free radical scavenger, an antiarrhythmic and has anti-inflammatory and anti-hypercoagulable properties. It must also be appreciated that at non-anaesthetic therapeutic concentrations, local anaesthetics like lignocaine would not completely block the voltage-dependent sodium fast channels, but would down-regulate channel activity and reduce sodium entry. As anti-arrhythmic, lignocaine is believed to target small sodium currents that normally continue through phase 2 of the action potential and consequently shortens the action potential and the refractory period.

As lignocaine acts by primarily blocking sodium fast channels, it will be appreciated that other sodium channel blockers may be used instead of or in combination with the antiarrhythmic agent in the composition of the present invention. It will also be appreciated that sodium channel blockers include compounds that act to substantially block sodium channels or at least downregulate sodium channels. Examples of suitable sodium channel blockers include venoms such as tetrodotoxin and the drugs primaquine, QX, HNS-32 (CAS Registry #186086-10-2), NS-7, kappa-opioid receptor agonist U50 488, crobenetine, pilsicamide, phenyloin, tocamide, mexiletine, NW-1029 (a benzylamino propanamide derivative), RS100642, riluzole, carbamazepine, flecainide, propafenone, amiodarone, sotalol, imipramine and moricizine, or any of derivatives thereof. Other suitable sodium channel blockers include: Vinpocetine (ethyl apovincaminate); and Beta-carboline derivative, nootropic beta-carboline (ambocarb, AMB).

In one embodiment, the composition according to the invention consists essentially of (i) a potassium channel opener or agonist and/or an adenosine receptor agonist; and (ii) an antiarrhythmic agent. Preferably, the antiarrhythmic agent is a local anaesthetic such as lignocaine.

In another embodiment of the invention, the composition according to the invention further includes an opioid. The inventor also found the inclusion of an opioid in the composition, particularly D-Pen[2,5]enkephalin (DPDPE), may also result in significantly less damage to the cell, tissue or organ.

Accordingly, in a further embodiment the composition according to the invention includes (i) a potassium channel opener or agonist and/or adenosine receptor agonist (ii) an antiarrhythmic agent and (iii) an opioid.

Opioids, also known or referred to as opioid agonists, are a group of drugs that inhibit opium (Gr opion, poppy juice) or morphine-like properties and are generally used clinically as moderate to strong analgesics, in particular, to manage pain, both peri- and post-operatively. Other pharmacological effects of opioids include drowsiness, respiratory depression, changes in mood and mental clouding without loss of consciousness.

Opioids are also believed to be involved as part of the ‘trigger’ in the process of hibernation, a form of dormancy characterised by a fall in normal metabolic rate and normal core body temperature. In this hibernating state, tissues are better preserved against damage that may otherwise be caused by diminished oxygen or metabolic fuel supply, and also protected from ischemia reperfusion injury.

There are three types of opioid peptides: enkephalin, endorphin and dynorphin.

Opioids act as agonists, interacting with stereospecific and saturable binding sites, in the heart, brain and other tissues. Three main opioid receptors have been identified and cloned, namely mu, kappa, and delta receptors. All three receptors have consequently been classed in the G-protein coupled receptors family (which class includes adenosine and bradykinin receptors). Opioid receptors are further subtyped, for example, the delta receptor has two subtypes, delta-1 and delta-2. Examples of opioid agonists include for example TAN-67, BW373U86, SNC80 ([(+)-4-[alpha(R)-alpha-[(2S,5R)-4-allyl-2,5-dimethyl-1-piperazinyl]-(3-methoxybenzyl)-N,N-diethylbenzamide), (+)BW373U86, DADLE, ARD-353 [4-((2R5S)-4-(R)-4-diethylcarbamoylphenyl)(3-hydroxyphenyl)methyl)-2,5-dimethylpiperazin-1-ylmethyl)benzoic acid], a nonpeptide delta receptor agonist, DPI-221 [4-((alpha-S)-alpha-((2S,5R)-2,5-dimethyl-4-(3-fluorobenzyl)-1-piperazinyl)benzyl)-N,N-diethylbenzamide].

Cardiovascular effects of opioids are directed within the intact body both centrally (ie, at the cardiovascular and respiratory centres of the hypothalamus and brainstem) and peripherally (ie, heart myocytes and both direct and indirect effects on the vasculature). For example, opioids have been shown to be involved in vasodilation. Some of the action of opioids on the heart and cardiovascular system may involve direct opioid receptor mediated actions or indirect, dose dependent non-opioid receptor mediated actions, such as ion channel blockade which has been observed with antiarrhythmic actions of opioids, such as arylacetamide drugs. It is also known that the heart is capable of synthesising or producing the three types of opioid peptides, namely, enkephalin, endorphin and dynorphin. However, only the delta and kappa opioid receptors have been identified on ventricular myocytes.

Without being bound by any mode of action, opioids are considered to provide cardioprotective effects, by limiting ischaemic damage and reducing the incidence of arrhythmias, which are produced to counter-act high levels of damaging agents or compounds naturally released during ischemia. This may be mediated via the activation of ATP sensitive potassium channels in the sarcolemma and in the mitochondrial membrane and involved in the opening potassium channels. Further, it is also believed that the cardioprotective effects of opioids are mediated via the activation of ATP sensitive potassium channels in the sarcolemma and in the mitochondrial membrane.

It will be appreciated that the opioids include compounds which act both directly and indirectly on opioid receptors. Opioids also include indirect dose dependent, non-opioid receptor mediated actions such as ion channel blockade which have been observed with the antiarrhythmic actions of opioids. Opioids and opioid agonists may be peptidic or non-peptidic. Preferably the opioid is selected from enkephalins, endorphins and dynorphins. Preferably, the opioid is an enkephalin which targets delta, kappa and/or mu receptors. More preferably the opioid is selected from delta-1-opioid receptor agonists and delta-2-opioid receptor agonists. D-Pen [2, 5]enkephalin (DPDPE) is a particularly preferred Delta-1-Opioid receptor agonist. In one embodiment, it is administered at 0.001 to 10 mg/kg body weight, preferably 0.01 to 5 mg/kg, or more preferably 0.1 to 1.0 mg/kg.

Accordingly, the present invention also relates to a method for reducing damage to a cell, tissue or organ following trauma comprising: administering to the cell, tissue or organ an effective amount of a composition including (i) a potassium channel opener or agonist and/or an adenosine receptor agonist; (ii) an antiarrhythmic agent; and (iii) an opioid; and postconditioning the cell, tissue or organ.

The present invention also relates to a method of reducing damage to a cell, tissue or organ following ischemia comprising: administering to the cell, tissue or organ an effective amount of a composition including (i) a potassium channel opener or agonist and/or an adenosine receptor agonist; (ii) an antiarrhythmic agent; and (iii) an opioid; and postconditioning the cell, tissue or organ.

The present invention also relates to a method of reducing damage to a cell, tissue or organ prior to or during ischemia or reperfusion comprising: administering to the cell, tissue or organ an effective amount of a composition including (i) a potassium channel opener or agonist and/or an adenosine receptor agonist; (ii) an antiarrhythmic agent; and (iii) an opioid; and postconditioning the cell, tissue or organ.

The inventor has also found that the injury may also be improved with this composition when administered without the postconditioning step.

Thus, in another aspect, the invention relates to a method of reducing damage to a cell, tissue or organ prior to or during ischemia or reperfusion comprising administering to the cell, tissue or organ an effective amount of a composition including (i) a potassium channel opener or agonist and/or adenosine receptor agonist; (ii) an antiarrhythmic agent and (iii) an opioid.

In another aspect, the invention also relates to a method of reducing damage to a cell, tissue or organ following ischemia comprising administering to the cell, tissue or organ an effective amount of a composition including (i) a potassium channel opener or agonist and/or adenosine receptor agonist; (ii) an antiarrhythmic agent and (iii) an opioid.

In yet another aspect, the invention relates to a method of reducing damage to a cell, tissue or organ following trauma comprising administering to the cell, tissue or organ an effective amount of a composition including (i) a potassium channel opener or agonist and/or adenosine receptor agonist; (ii) an antiarrhythmic agent and (iii) an opioid.

In some embodiments the composition useful in the methods according to the invention may include additional potassium channel openers or agonists, for example, diazoxide, or nicorandil.

Diazoxide is a potassium channel opener and in the present invention it is believed to preserve ion and volume regulation, oxidative phosphorylation and mitochondrial membrane integrity (appears concentration dependent). More recently, diazoxide has been shown to provide cardioprotection by reducing mitochondrial oxidant stress at reoxygenation. At present it is not known if the protective effects of potassium channel openers are associated with modulation of reactive oxygen species generation in mitochondria. Preferably the concentration of the diazoxide is between about 1 to 200 uM. Typically this is as an effective amount of diazoxide. More preferably, the concentration of diazoxide is about 10 uM.

Nicorandil is a potassium channel opener and nitric oxide donor which can protect tissues and the microvascular integrity including endothelium from ischemia and reperfusion damage. Thus it can exert benefits through the dual action of opening KATP channels and a nitrate-like effect. Nicorandil can also reduce hypertension by causing blood vessels to dilate which allows the heart to work more easily by reducing both preload and afterload. It is also believed to have anti-inflammatory and anti-proliferative properties which may further attenuate ischemia/reperfusion injury.

The composition useful in the methods according to the invention may further include at least one compound for minimizing or reducing the uptake of water by a cell in the cell, tissue or organ.

A compound for minimizing or reducing the uptake of water by a cell in the tissue tends to control water shifts, ie, the shift of water between the extracellular and intracellular environments. Accordingly, these compounds are involved in the control or regulation of osmosis. One consequence is that a compound for minimizing or reducing the uptake of water by a cell in the tissue reduces cell swelling that is associated with Oedema, such as Oedema that can occur during ischemic injury.

Compounds for minimizing or reducing the uptake of water by a cell in a tissue are typically impermeants or receptor antagonists or agonists. An impermeant according to the present invention may be selected from one or more of the group consisting of: sucrose, pentastarch, hydroxyethyl starch, raffinose, mannitol, gluconate, lactobionate, and colloids. Colloids include albumin, hetastarch, polyethylene glycol (PEG), Dextran 40 and Dextran 60. Other compounds that could be selected for osmotic purposes include those from the major classes of osmolytes found in the animal kingdom including polyhydric alcohols (polyols) and sugars, other amino acids and amino-acid derivatives, and methylated ammonium and sulfonium compounds.

Cell swelling can also result from an inflammatory response which may be important during organ retrieval, preservation and surgical grafting. Substance P, an important pro-inflammatory neuropeptide is known to lead to cell oedema and therefore antagonists of substance P may reduce cell swelling. Indeed antagonists of substance P, (-specific neurokinin-1) receptor (NK-1) have been shown to reduce inflammatory liver damage, i.e., oedema formation, neutrophil infiltration, hepatocyte apoptosis, and necrosis. Two such NK-1 antagonists include CP-96,345 or [(2S,3S)-cis-2-(diphenylmethyl)-N-((2-methoxyphenyl)-methyl)-1-azabicyclo[2.2.2.)-octan-3-amine (CP-96,345)] and L-733,060 or [(2S,3S)3-([3,5-bis(trifluoromethyl)phenyl]methoxy)-2-phenylpiperidine]. R116301 or [(2R-trans)-4-[1-[3,5-bis(trifluoromethyl)benzoyl]-2-(phenylmethyl)-4-piperidinyl]-N-(2,6-dimethylphenyl)-1-acetamide (S)-Hydroxybutanedioate] is another specific, active neurokinin-1 (NK(1)) receptor antagonist with subnanomolar affinity for the human NK(1) receptor (K(i): 0.45 nM) and over 200-fold selectivity toward NK(2) and NK(3) receptors. Antagonists of neurokinin receptors 2 (NK-2) that may also reduce cell swelling include SR48968 and NK-3 include SR142801 and SB-222200. Blockade of mitochondrial permeability transition and reducing the membrane potential of the inner mitochondrial membrane potential using cyclosporin A has also been shown to decrease ischemia-induced cell swelling in isolated brain slices. In addition glutamate-receptor antagonists (AP5/CNQX) and reactive oxygen species scavengers (ascorbate, Trolox(R), dimethylthiourea, tempol(R)) also showed reduction of cell swelling. Thus, the compound for minimizing or reducing the uptake of water by a cell in a tissue can also be selected from any one of these compounds.

It will also be appreciated that the following energy substrates can also act as impermeants. Suitable energy substrate can be selected from one or more from the group consisting of: glucose and other sugars, pyruvate, lactate, glutamate, glutamine, aspartate, arginine, ectoine, taurine, N-acetyl-beta-lysine, alanine, proline, beta-hydroxy butyrate and other amino acids and amino acid derivatives, trehalose, floridoside, glycerol and other polyhydric alcohols (polyols), sorbitol, myo-innositol, pinitol, insulin, alpha-keto glutarate, malate, succinate, triglycerides and derivatives, fatty acids and carnitine and derivatives. In one embodiment, the at least one compound for minimizing or reducing the uptake of water by the cells in the tissue is an energy substrate. The energy substrate helps with recovering metabolism. The energy substrate can be selected from one or more from the group consisting of: glucose and other sugars, pyruvate, lactate, glutamate, glutamine, aspartate, arginine, ectoine, taurine, N-acetyl-beta-lysine, alanine, proline and other amino acids and amino acid derivatives, trehalose, floridoside, glycerol and other polyhydric alcohols (polyols), sorbitol, myo-innositol, pinitol, insulin, alpha-keto glutarate, malate, succinate, triglycerides and derivatives, fatty acids and carnitine and derivatives. Given that energy substrates are sources of reducing equivalents for energy transformations and the production of ATP in a cell, tissue or organ of the body, it will be appreciated that a direct supply of the energy reducing equivalents could be used as substrates for energy production. For example, a supply of either one or more or different ratios of reduced and oxidized forms of nicotinamide adenine dinucleotide (e.g. NAD or NADP and NADH or NADPH) or flavin adenine dinucleotides (FADH or FAD) could be directly used to supply bond energy for sustaining ATP production in times of stress. Preferably, beta-hydroxy butyrate is added to the composition of the invention for treatment of trauma or reducing injury.

In addition to providing energy substrates to the whole body, organ, tissue or cell, improvements in metabolising these substrates may occur in the presence of hydrogen sulphide (H₂S) or H₂S donors (eg NaHS). The presence of hydrogen sulphide (H₂S) or H2S donors (eg NaHS) may help metabolise these energy substrates by lowering energy demand during arrest, protect and preserve the whole body, organ, tissue or cell during periods of metabolic imbalance such ischemia, reperfusion and trauma. Concentrations of hydrogen sulfide above 1 microM (10-6 M) concentration can be a metabolic poison that inhibits respiration at Respiratory Complex IV, which is part of the mitochondrial respiratory chain that couples metabolising the high energy reducing equivalents from energy substrates to energy (ATP) generation and oxygen consumption. However, it has been observed at lower concentrations, below 10-⁶ M (eg 10-¹⁰ to 10-⁹M), hydrogen sulfide may reduce the energy demand of the whole body, organ, tissue or cell which may result in arrest, protection and preservation. In other words, very low levels of sulfide down-regulate mitochondria, reduce O₂ consumption and actually increase “Respiratory Control” whereby mitochondria consume less O₂ without collapsing the electrochemical gradient across the inner mitochondrial membrane. Thus there are observations that a small amount of sulfide, either directly or indirectly, may close proton leak channels and better couple mitochondrial respiration to ATP production more tightly, and this effect may improve the metabolism of high energy reducing equivalents from energy substrates. There is also the possibility that a sulphur cycle exists between the cell cytosol and mitochondria in mammals, including humans, providing the sulphur concentration is low. The presence of a vestige sulphur cycle would be consistent with current ideas on the evolutionary origin of mitochondria and their appearance in eukaryote cells from a symbiosis between a sulfide-producing host cell and a sulfide-oxidizing bacterial symbiont. Thus, hydrogen sulphide (H₂S) or H₂S donors (eg NaHS) may be energy substrates themselves in addition to improving the metabolism of other energy substrates. Accordingly, in one form, the invention provides a composition as described above further including hydrogen sulphide or a hydrogen sulfide donor.

Preferably, the compound for minimizing or reducing the uptake of water by the cells in the tissue is sucrose. Sucrose reduces water shifts as an impermeant. Impermeant agents such as sucrose, lactobionate and raffinose are too large to enter the cells and hence remain in the extracellular spaces within the tissue and resulting osmotic forces prevent cell swelling that would otherwise damage the tissue, which would occur particularly during storage of the tissue.

In another embodiment, the at least one compound for minimizing or reducing the uptake of water by the cells in the tissue is a colloid. Suitable colloids include, but not limited to, Dextran-70, 40, 50 and 60, hydroxyethyl starch and a modified fluid gelatin. A colloid is a composition which has a continuous liquid phase in which a solid is suspended in a liquid. Colloids can be used clinically to help restore balance to water and ionic distribution between the intracellular, extracellular and blood compartments in the body after an severe injury. Colloids can also be used in solutions for organ preservation. Administration of crystalloids can also restore water and ionic balance to the body but generally require greater volumes of administration because they do not have solids suspended in a liquid. Thus volume expanders may be colloid-based or crystalloid-based

Preferably, the concentration of the compound for minimizing or reducing the uptake of water by the cells in the tissue is between about 5 to 500 mM. Typically this is an effective amount for reducing the uptake of water by the cells in the tissue. More preferably, the concentration of the compound for reducing the uptake of water by the cells in the tissue is between about 20 and 100 mM. Even more preferably the concentration of the compound for reducing the uptake of water by the cells in the tissue is about 70 mM.

In a further embodiment, the composition useful in the methods according to the invention may include more than one compound for minimizing or reducing the uptake of water by the cells in the tissue. For example, a combination of impermeants (raffinose, sucrose and pentastarch) may be included in the composition or even a combination of colloids, and fuel substrates may be included in the composition.

The inventor has also found that the inclusion of a compound for inhibiting transport of sodium and hydrogen ions across a plasma membrane of a cell in the tissue with a potassium channel opener or agonist and/or adenosine receptor agonist and an antiarrhythmic agent assists in reducing injury and damage.

Thus in another aspect, the composition useful in the methods according to the invention further includes a compound for inhibiting transport of sodium and hydrogen ions across a plasma membrane of a cell in the tissue.

The compound for inhibiting transport of sodium and hydrogen across the membrane of the cell in the tissue is also referred to as a sodium hydrogen exchange inhibitor. The sodium hydrogen exchange inhibitor reduces sodium and calcium entering the cell.

Preferably the compound for inhibiting transport of sodium and hydrogen across the membrane of the cell in the tissue may be selected from one or more of the group consisting of Amiloride, EIPA(5-(N-entyl-N-isopropyl)-amiloride), cariporide (HOE-642), eniporide, Triamterene (2,4,7-triamino-6-phenylteride), EMD 84021, EMD 94309, EMD 96785, EMD 85131, HOE 694. B11 B-513 and T-162559 are other inhibitors of the isoform 1 of the Na+/H+ exchanger.

Preferably, the sodium hydrogen exchange inhibitor is Amiloride (N-amidino-3,5-diamino-6-chloropyrzine-2-carboximide hydrochloride dihydrate). Amiloride inhibits the sodium proton exchanger (Na+/H+ exchanger also often abbreviated NHE-1) and reduces calcium entering the cell. During ischemia excess cell protons (or hydrogen ions) are believed to be exchanged for sodium via the Na+/H+ exchanger.

Preferably, the concentration of the sodium hydrogen exchange inhibitor is between about 1.0 nM to 1.0 mM. More preferably, the concentration of the sodium hydrogen exchange inhibitor in the tissue is about 20 μM.

The composition useful in the methods according to the invention may also include an antioxidant. Antioxidants are commonly enzymes or other organic substances that are capable of counteracting the damaging effects of oxidation in the tissue. The antioxidant component of the composition according to the present invention may be selected from one or more of the group consisting of: allopurinol, carnosine, histidine, Coenzyme Q 10, n-acetyl-cysteine, superoxide dismutase (SOD), glutathione reductase (GR), glutathione peroxidase (GP) modulators and regulators, catalase and the other metalloenzymes, NADPH and NAD(P)H oxidase inhibitors, glutathione, U-74006F, vitamin E, Trolox (soluble form of vitamin E), other tocopherols (gamma and alpha, beta, delta), tocotrienols, ascorbic acid, Vitamin C, Beta-Carotene (plant form of vitamin A), selenium, Gamma Linoleic Acid (GLA), alpha-lipoic acid, uric acid (urate), curcumin, bilirubin, proanthocyanidins, epigallocatechin gallate, Lutein, lycopene, bioflavonoids, polyphenols, trolox(R), dimethylthiourea, tempol(R), carotenoids, coenzyme Q, melatonin, flavonoids, polyphenols, aminoindoles, probucol and nitecapone, 21-aminosteroids or lazaroids, sulphydryl-containing compounds (thiazolidine, Ebselen, dithiolethiones), and N-acetylcysteine. Other antioxidants include the ACE inhibitors (captopril, enalapril, lisinopril) which are used for the treatment of arterial hypertension and cardiac failure on patients with myocardial infarction. ACE inhibitors exert their beneficial effects on the reoxygenated myocardium by scavenging reactive oxygen species. Other antioxidants that could also be used include beta-mercaptopropionylglycine, 0-phenanthroline, dithiocarbamate, selegilize and desferrioxamine (Desferal), an iron chelator, has been used in experimental infarction models, where it exerted some level of antioxidant protection. Spin trapping agents such as 5′-5-dimethyl-1-pyrrolione-N-oxide (DMPO) and (a-4-pyridyl-1-oxide)-N-t-butylnitrone (POBN) also act as antioxidants. Other antioxidants include: nitrone radical scavenger alpha-phenyl-tert-N-butyl nitrone (PBN) and derivatives PBN (including disulphur derivatives); N-2-mercaptopropionyl glycine (MPG) a specific scavenger of the OH free radical; lipooxygenase inhibitor nordihydroguaretic acid (NDGA); Alpha Lipoic Acid; Chondroitin Sulfate; L-Cysteine; oxypurinol and Zinc.

Preferably, the antioxidant is allopurinol (1H-Pyrazolo[3,4-α]pyrimidine-4-ol). Allopurinol is a competitive inhibitor of the reactive oxygen species generating enzyme xanthine oxidase. Allopurinol's antioxidative properties may help preserve myocardial and endothelial functions by reducing oxidative stress, mitochondrial damage, apoptosis and cell death.

The inventor has also found that the inclusion of particular amounts of calcium and magnesium ions with a potassium channel opener or agonist and/or adenosine receptor agonist and an antiarrhythmic agent may also reduce injury. The effect of the particular amounts of calcium and magnesium ions is to control the amount of ions within the intracellular environment. Calcium ions tend to be depleted, exported or otherwise removed from the intracellular environment and magnesium ions tend to be increased or otherwise restored to the levels typically found in a viable, functioning cell.

Thus in another aspect, the composition useful in the methods according to the invention may further include a source of magnesium in an amount for increasing the amount of magnesium in a cell in body tissue. Preferably the magnesium is present at a concentration of between 0.5 mM to 20 mM, more preferably about 2.5 mM.

In addition, typical buffers or carriers (which are discussed in more detail below) in which the composition of the invention is administered typically contain calcium at concentrations of around 1 mM as the total absence of calcium has been found to be detrimental to the cell, tissue or organ. In one form, the invention may also include using carriers with low calcium (such as for example less than 0.5 mM) so as to decrease the amount of calcium within a cell in body tissue, which may otherwise build up during injury/trauma/stunning. Preferably the calcium present is at a concentration of between 0.1 mM to 0.8 mM, more preferably about 0.3 mM. As described in the present invention, elevated magnesium and low calcium has been associated with protection during ischemia and reoxygenation of an organ. The action is believed to be due to decreased calcium loading.

In one embodiment, the composition useful in the methods according to the invention includes elevated magnesium ions i.e. over normal plasma concentrations. Preferably the magnesium is divalent and present at a concentration of between 0.5 mM to 20 mM, more preferably about 16 mM. Magnesium sulphate and magnesium chloride is a suitable source.

In a further aspect, the composition useful in the methods according to the invention includes an antiarrhythmic agent and one or more of:
potassium channel opener or agonist;
adenosine receptor agonist;
opioid;
calcium channel blocker;
at least one compound for reducing uptake of water;
sodium hydrogen exchange inhibitor;
antioxidant; and
a source of magnesium in an amount for increasing the amount of magnesium in a cell in body tissue.

Preferably, this composition has two, three or four of the above components. Preferred compounds for these components are listed above. It is also contemplated that this composition may include more than one of the same component, for example two different potassium channel openers may be present in the composition. It is also contemplated that one component may have more than one function. For example, some calcium antagonists share effects with potassium channel openers.

In another aspect there is also provided a composition useful in the methods according to the invention further including an effective amount of elevated magnesium.

In one embodiment the composition useful in the methods according to the invention includes adenosine and lignocaine. This composition may optionally include an elevated source of magnesium and/or opioid. Preferably, the opioid is a delta opioid, such as DPDPE.

In another embodiment the composition useful in the methods according to the invention includes CCPA and lignocaine. This composition may optionally include an elevated source of magnesium and/or opioid. Preferably, the opioid is a delta opioid, such as DPDPE.

The processes of inflammation and thrombosis are linked through common mechanisms. Therefore, it is believed that understanding of the processes of inflammation will help with better management of thrombotic disorders including the treatment of acute and chronic ischaemic syndromes. In the clinical and surgical settings, a rapid response and early intervention to an organ or tissue damaged from ischemia can involve both anti-inflammatory and anti-clotting therapies. In addition to protease inhibitors which attenuate the inflammatory response, further anti-inflammatory therapies have included the administration of aspirin, normal heparin, low-molecular-weight heparin (LMWH), non-steroidal anti-inflammatory agents, anti-platelet drugs and glycoprotein (GP) IIb/IIIa receptor inhibitors, statins, angiotensin converting enzyme (ACE) inhibitor, angiotensin blockers and antagonists of substance P. Examples of protease inhibitors are indinavir, nelfinavir, ritonavir, lopinavir, amprenavir or the broad-spectrum protease inhibitor aprotinin, a low-molecular-weight heparin (LMWH) is enoxaparin, non-steroidal anti-inflammatory agent are indomethacin, ibuprofen, rofecoxib, naproxen or fluoxetine, an anti-platelet drug such as aspirin, a glycoprotein (GP) IIb/IIIa receptor inhibitor is abciximab, a statin is pravastatin, an angiotensin converting enzyme (ACE) inhibitor is captopril and an angiotensin blocker is valsartin.

Accordingly, in another embodiment of the invention, a selection of these agents is added to the composition useful in the methods according to the invention to deliver improved management of inflammation and clotting in order to reduce injury to cells, tissues or organs. Alternatively, the composition according to the invention may be administered together with any one or more of these agents.

In particular, protease inhibitors attenuate the systemic inflammatory response in patients undergoing cardiac surgery with cardiopulmonary bypass, and other patients where the inflammatory response has been heightened such as AIDS or in the treatment of chronic tendon injuries. Some broad spectrum protease inhibitors such as aprotinin are also reduce blood loss and need for blood transfusions in surgical operations such as coronary bypass.

Compounds that substantially prevent the breakdown of adenosine in the blood such as nucleoside transport inhibitors, such as dipyridamole could be are used as additives in the composition of the invention. The half life of adenosine in the blood is about 10 seconds so the presence of a medicament to substantially prevent its breakdown will maximise the effect of the composition of the present invention.

In another embodiment, the composition useful in the methods according to the invention includes a cellular transport enzyme inhibitor, such as dipyridamole, to prevent metabolism or breakdown of components in the composition.

Dipyridamole is advantageously included in a concentration from about 0.01 microM to about 10 mM, preferably 0.05 to 100 μM., Dipyridamole and has major advantages with respect to cardioprotection. Dipyridamole may supplement the actions of adenosine by inhibiting adenosine transport and breakdown leading to increased protection of cells, tissues and organs of the body during times of stress. Dipyridamole may also be administered separately for example by 400 mg daily tablets to produce a plasma level of about 0.4 μg/ml, or 0.8 μM concentration.

The composition useful in the methods of the invention is highly beneficial at about 10° C. but can also be used to prevent injury over a wider temperature range up to about 37° C. Accordingly, the composition may be administered to the cell, tissues or organs at a temperature range selected from one of the following: from about 0° C. to about 5° C., from about 5° C. to about 20° C., from about 20° C. to about 32° C. and from about 32° C. to about 38° C. It is understood that “profound hypothermia” is used to describe a tissue at a temperature from about 0° C. to about 5° C. “Moderate hypothermia” is used to describe a tissue at a temperature from about 5° C. to about 20° C. “Mild hypothermia” is used to describe a tissue at a temperature from about 20° C. to about 32° C. “Normothermia” is used to describe a tissue at a temperature from about 32° C. to about 38° C., though the normal body temperature is around 37 to 38° C.

In another embodiment, the composition useful in the methods according to the invention may be administered with or contain blood or blood products or artificial blood or oxygen binding molecules or solutions to improve the body's oxygen transport ability and survival by helping to reduce hypoxic and ischemic damage from blood loss. The oxygen-containing molecules, compounds or solutions may be selected from natural or artificial products. For example, an artificial blood-based product is perfluorocarbon-based or other haemoglobin-based substitute. Some of the components may be added to mimic human blood's oxygen transport ability such Hemopure™, Gelenpol™, Oxygent™, and PolyHeme™. Hemopore is based on a chemically stabilized bovine hemoglobin. Gelenpol is a polymerized hemoglobin which comprises synthetic water-soluble polymers and modified heme proteins. Oxygent is a perflubron emulsion for use as an intravenous oxygen carrier to temporarily substitute for red blood cells during surgery. Polyheme is a human hemoglobin-based solution for the treatment of life-threatening blood loss.

It is believed that the oxygenation of the body from a variety of ways including but not limited to oxygen gas mixture, blood, blood products or artificial blood or oxygen binding solutions maintains mitochondrial oxidation and this helps preserve the myocyte and endothelium of the organ. Without being bound by any particular mode or theory, the inventor has found that gentle bubbling with 95% O₂/5% CO₂ helps maintains mitochondrial oxidation which helps preserve the myocyte and coronary vasculature.

In one preferred embodiment the composition useful in the methods according to the invention is aerated with a source of oxygen before and/or during administration. The source of oxygen may be an oxygen gas mixture where oxygen is the predominant component.

In another aspect of the present invention there is provided a method for reducing injury to a cell, tissue or organ including:
providing in a suitable container a composition as described above;
providing one or more nutrient molecules selected from the group consisting of blood, blood products, artificial blood and a source of oxygen;
optionally aerating the composition with the oxygen (for example, in the case of isolated organs) or combining the nutrient molecules with the composition, or both; and
placing the tissue, cell or organ in contact with the combined composition under conditions sufficient to reduce injury.

This method may include the further step of postconditioning the cell, tissue or organ.

Preferably the oxygen source is an oxygen gas mixture. Preferably oxygen is the predominant component. The oxygen may be mixed with, for example CO₂. More preferably, the oxygen gas mixture is 95% O₂ and 5% CO₂.

The composition may be suitable for administration to the tissue in liquid form, for example, solutions, syrups or suspensions, or alternatively they may be administered as a dry product for constitution with water or other suitable vehicle before use. Alternatively, the composition may be presented as a dry product for constitution with water or other suitable vehicle. Such liquid preparations may be prepared by conventional means with pharmaceutically acceptable additives such as suspending agents, emulsifying agents, non-aqueous vehicles, preservatives and energy sources. In another form, the invention comprises a composition in tablet form and in another form, the invention comprises an aerosol which could be administered via oral, skin or nasal routes.

The composition useful in the methods according to the invention may be suitable for topical administration to the tissue. Such preparation may be prepared by conventional means in the form of a cream, ointment, jelly, solution or suspension.

The composition may also be formulated as depot preparations. Such long acting formulations may be administered by implantation (eg, subcutaneously or intramuscularly) or by intramuscular injection. Thus, for example, the composition according to the invention may be formulated with suitable polymeric or hydrophobic materials (eg, as an emulsion in an acceptable oil or ion exchange resins, or as sparingly soluble derivatives, for example, as a sparingly soluble salt.

The method of the present invention involves contacting a tissue with the composition for a time and under conditions sufficient for reducing injury to the cell, tissue or organ. The composition may for example be infused or administered as a bolus intravenous, intracoronary or any other suitable delivery route as pre-treatment for protection during a cardiac intervention such as open heart surgery (on-pump and off-pump), angioplasty (balloon and with stents or other vessel devices) and as with clot-busters (anti-clotting drug or agents).

The composition may be administered intravenously or be administered both intravenously and intraperitoneally or directly accessing a major artery such as the femoral artery or aorta in patients who have no pulse from massive exsanguination, or in the carotid artery or another artery during aortic dissection to protect the brain from hypoxia or ischemia. In one embodiment, the composition may be administered intravenously and intraperineally simultaneously, the perineum acting as, in effect, a reservoir of composition for the bloodstream as well as acting on organs in the vicinity with which it comes into contact. This is particularly suitable for a trauma victim, such as one suffering shock. Moreover, where the composition contains two or more components, these may be administered separately but simultaneously. Substantially simultaneous delivery of the component to the target site is desirable. This may be achieved by pre-mixing the components for administration as one composition, but that is not essential.

The invention is directed towards the simultaneous increase in local concentration (for example an organ such as the heart) of the components of a composition (for example, where a first component is (i) a potassium channel opener or agonist and/or an adenosine receptor agonist; and (ii) an antiarrhythmic agent).

One preferred form of the composition is a combination of adenosine and lignocaine. In another preferred form, the composition may also include an opioid, preferably a delta-1-opioid receptor against, such as DPDPE.

The invention may be practised by administering the composition using a perfusion pump, often associated with a procedure known as “miniplegia” or “microplegia”, in which minimal amount of components are titrated by means of a finely adjustable pump directly via a catheter. In the invention, a protocol utilises miniplegia as described above, where micro amounts are titrated directly to the heart, using the patient's own oxygenated blood. The reference to a “setting” is a measure on the pump, such as a syringe pump, of the amount of substance being delivered directly to the organ, such as a heart.

Alternatively, the composition may be administered by aerosol.

The composition can also be infused or administered as a bolus intravenous, intracoronary or any other suitable delivery route for protection during cardiac intervention such as open heart surgery (on-pump and off-pump), angioplasty (balloon and with stents or other vessel devices) and as with clot-busters to protect and preserve the cells from injury.

Accordingly, the tissue may be contacted by delivering the composition intravenously to the tissue. This involves using blood as a vehicle for delivery to the tissue. In particular, the composition may be used for blood cardioplegia. Alternatively, the composition may be administered directly as a bolus by a puncture (eg, by syringe) directly to the tissue or organ, particularly useful when blood flow to a tissue or organ is limiting. The composition for arresting, protecting and preserving a tissue may also be administered as an aerosol, powder, solution or paste via oral, skin or nasal routes.

Alternatively, the composition may be administered directly to the tissue, organ or cell or to exposed parts of the internal body to reduce injury.

The composition according to the invention may be used with crystalloid cardioplegia to minimise injury to a tissue. In one application for a surgical or diagnostic procedure such a composition could be administered to provide localised arrest of the target tissue as well as protection during reperfusion and postconditioning.

The composition may be delivered according to one of or a combination of the following delivery protocols: intermittent, continuous and one-shot. Accordingly, in another aspect of the invention, the composition may be administered as a single dose of the composition.

In another aspect of the invention, the composition may be administered by intermittent administration. A suitable administration schedule is a 2 minute induction dose every 20 minutes throughout the arrest period. The actual time periods can be adjusted based on observations by one skilled in the art administering the composition, and the animal/human model selected. The invention also provides a method for intermittently administering a composition for reducing injury to the cell, tissue or organ.

The composition can of course also be used in continuous infusion with both normal and injured tissues or organs, such as heart tissue. Continuous infusion also includes static storage of the tissue, whereby the tissue is stored in a composition according to the invention, for example the tissue may be placed in a suitable container and immersed in a composition (or solution) for transporting donor tissues from a donor to recipient.

The dose and time intervals for each delivery protocol may be designed accordingly. The components of the composition according to the invention may be combined prior to administration or administered substantially simultaneously or co-administered.

While it is possible for each component of the composition to contact the tissue alone, it is preferable that the components of the pharmaceutical composition be provided together with one or more pharmaceutically acceptable carriers, diluents, adjuvants and/or excipients. Each carrier, diluent, adjuvant and/or excipient must be pharmaceutically acceptable such that they are compatible with the components of the pharmaceutical composition and not harmful to the subject. Preferably, the pharmaceutical composition is prepared with liquid carriers, diluents, adjuvants and/or excipients.

Aqueous suspensions contain the active materials in admixture with excipients suitable for the manufacture of aqueous suspensions. Such excipients are suspending agents, for example sodium carboxymethylcellulose, methylcellulose, hydropropyl methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. Aqueous suspensions may also contain one or more preservatives, for example benzoates, such as ethyl, or n-propyl p-hydroxybenzoate, one or more colouring agents, one or more flavouring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Syrups and elixirs may be formulated with sweetening agents, for example glycerol, propylene glycol, sorbitol or sucrose. Such formulations may also contain a demulcent, a preservative and flavouring and colouring agents.

Accordingly, this aspect of the invention also provides a composition useful in the methods according to the invention together with a pharmaceutically acceptable carrier, diluent, adjuvant and/or excipient. A preferred pharmaceutically acceptable carrier is a buffer having a pH of about 6 to about 9, preferably about 7, more preferably about 7.4 and/or low concentrations of potassium. For example, the composition has a total potassium concentration of up to about 10 mM, more preferably about 2 to about 8 mM, most preferably about 4 to about 6 mM. Suitable buffers include Krebs-Henseleit which generally contains 10 mM glucose, 117 mM NaCl, 5.9 mM KCl, 25 mM NaHCO₃, 1.2 mM NaH₂PO₄, 1.12 mMCaCl₂ (free Ca²⁺=1.07 mM) and 0.512 mM MgCl₂ (free Mg²⁺=0.5 mM), Tyrodes solution which generally contains 10 mM glucose, 126 mM NaCl, 5.4 mM KCl, 1 mM CaCl₂, 1 mM MgCl₂, 0.33 mM NaH₂PO₄ and 10 mM HEPES (N-[2-hydroxyethyl]piperazine-N′-[2-ethane sulphonic acid], Fremes solution, Hartmanns solution which generally contains 129 NaCl, 5 mM KCl, 2 mM CaCl₂ and 29 mM lactate and Ringers-Lactate.

Other naturally occurring buffering compounds that exist in muscle that could be also used in a suitable ionic environment are carnosine, histidine, anserine, ophidine and balenene, or their derivatives.

It is also advantageous to use carriers having low concentrations of magnesium, such as, for example up to about 2.5 mM, but it will be appreciated that high concentrations of magnesium, for example up to about 20 mM, may be used if desired without substantially affecting the activity of the composition.

It will be appreciated that the concentrations of each component in the composition may be diluted by body fluids or other fluids that may be administered together with the composition. Typically, the composition will be administered such that the concentration of each component in the composition contacts the tissue about 100-fold less. For example, containers such as vials that house the composition may be diluted 1 to 100 parts of blood, plasma, crystalloid or blood substitute for administration.

In another aspect of the present invention there is provided use of a composition as described above for the preparation of a medicament for reducing injury of a cell, tissue or organ. In one embodiment of this aspect of the invention, there is provided use of a composition as described above for the preparation of a medicament for reducing damage to a cell, tissue or organ following ischemia.

In another embodiment, there is provided use of a composition as described above for the preparation of a medicament for reducing damage to a cell, tissue or organ following trauma.

In a further embodiment, there is provided use of a composition as described above for the preparation of a medicament for reducing damage to a cell, tissue or organ prior to or during ischemia or reperfusion.

It will be understood that the invention disclosed and defined in this specification extends to all alternative combinations of two or more of the individual features mentioned or evident from the text or drawings. All of these different combinations constitute various alternative aspects of the invention.

### FIGURE(S)

FIG. 1: Bar graph showing infarct size reduction using: Saline iv infusion alone (Control); lidocaine iv infusion alone (L); Adenosine and lidocaine iv infusion (AL); Adenosine iv infusion alone (A); Post-conditioning alone (PC); Adenosine and lidocaine iv infusion+post conditioning (AL+PC); Adenosine and lidocaine iv infusion+Opioid agonist DPDPE (AL+DPDPE). Numbers in parentheses are number of animals tested.

### EXAMPLES

The following are provided as non-limiting examples of suitable compositions and methods of the invention for the purpose of illustrating the invention.

### Example 1

### Adenosine+Lignocaine (AL) Therapy, and Postconditioning (PC)

This example examines intermediate doses of Adenosine+Lignocaine (AL) therapy, and effect of postconditioning (PC) induced seconds after reperfusion compared to AL alone and postconditioning alone on infarct size.

Postconditioning was discovered in 2003 by Vinten-Johansen and colleagues and is defined as rapid intermittent interruptions of blood flow in the early phase of reperfusion. Postconditioning may be applicable in the “off-pump” and “on-pump” surgery as well as angioplasty because the reperfusion can be controlled by the surgeon or the interventionist. Indeed, three clinical trials have now shown postconditioning during angioplasty to be effective in reducing infarct size by 30%, even as far as 7 days from the procedure.

Delta opioid receptor agonists are cardioprotective and strong analgesics with relatively few side effects. This example examines whether the presence of Delta-1-opioid receptor agonists e.g. [D-Pen-2,5] enkephalin (DPDPE) augments adenosine's effect in AL.

Experiments were performed on anaesthetised rats in vivo and regional ischemia was imposed by tying and ligating the left coronary artery for 30 min followed by 2 hrs of reperfusion. Drugs were administered intravenously 5 min before and during 30 min ischemia (see methods described above).

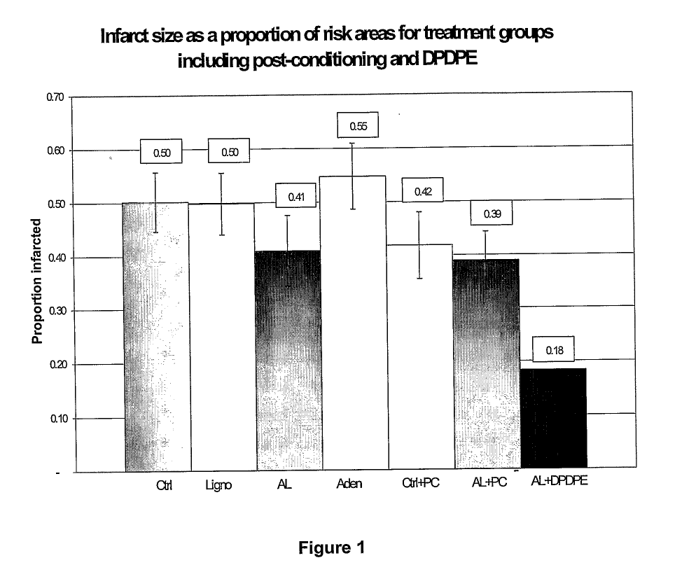

Data showing infarct size reduction using AL(305/60) (↓19%), AL(305/60)+postconditioning (↓32%), and AL(305/60)+DPDPE(↓64%) at the lower lignocaine concentration (60 umol/kg/min) are shown in FIG. 1. The numbers in parenthesis indicate concentrations of A and L respectively in ug/min/kg.

Although postconditioning (PC) did not significantly reduce infarct size in this experiment following adenosine and low lidocaine (305/60), it did dramatically reduce the number of reperfusion arrhythmias.

The data using AL plus delta-opioid DPDPE showed a marked reduction in infarct size from 50% to 18% (n=1) and again a marked fall in the incidence of arrhythmias. Without being bound any theory or mode of action, the addition of DPDPE may confer greater protection when combined with AL therapy.

### Example 2

### Effect of AL Plus Opioids and/or Post-Conditioning

This example illustrates AL's cardioprotective properties using ‘intermediate’ intravenous adenosine and lignocaine levels and the possible additive protection from postconditioning and possible cross-talk with delta opioid receptors. Postconditioning and opioid crosstalk is best analysed in the AL group that provides optimal protection.

In this example intravenous AL is infused 5 min before ligating the left coronary artery and continued during 30 min regional ischaemia. Four combinations of AL are studied: 300/120, 300/180, 150/120, 150/180 (A/L ug/min/kg) respectively.

AL's cardioprotective properties during 30 min CA ligation and 120 min reperfusion is examined. Rats are randomly assigned to one of 13 groups:

1) Saline controls (n=8).
2) AL-treated rats (A: 300 μg/kg/min plus L: 120 μg/kg/min administered intravenously 5 min before and during 30 min coronary artery ligation. (n=8).
3) AL-treated rats (A: 300 μg/kg/min plus L: 180 μg/kg/min administered intravenously 5 min before and during 30 min coronary artery ligation. (n=8).
4) AL-treated rats (A: 150 μg/kg/min plus L: 120 μg/kg/min administered intravenously 5 min before and during 30 min coronary artery ligation. (n=8).
5) AL-treated rats (A: 150 μg/kg/min plus L: 180 μg/kg/min administered intravenously 5 min before and during 30 min coronary artery ligation. (n=8).
6) A lido bolus and lido infusion treatment group (120 μg/kg/min) (n=8).
7) A lido bolus and lido infusion treatment group (180 μg/kg/min) (n=8).
8) Adenosine infusion (300 μg/kg/min) (n=8).
9) Adenosine infusion (150 μg/kg/min) (n=8).
10) Postconditioning (PC) (3 cycles of 10 sec occlusion and reperfusion beginning 10 sec after removing snare) (n=8) (Vinten-Johansen).
11) AL (300/120; 300/180; 150/120; 150/180)+PC (n=8).
12) AL (300/120; 300/180; 150/120; 150/180)+6 mg/kg i.v. naloxane administered 50 min before occlusion (n=8) [Ludwig, 2003 #1744].
13) AL (300/120; 300/180; 150/120; 150/180)+1.0 mg/kg [D-Pen-2,5]enkephalin (DPDPE) (11 min before occlusion) [Peart, 2003 #1575] (Gross) (n=8).
14) A1 receptor agonist (CCPA) with Lignocaine (n=8).
15) AL (300/120; 300/180; 150/120; 150/180)+1.0 mg/kg [D-Pen-2,5]enkephalin (DPDPE)+PC (n=8)

Total number of rats=120. A further 40 rats are used as saline-controls or A-alone or mistakes in ligating the left coronary artery. Previous studies show that about 50% of the saline-controls and A-alone animals die during 30 min ischaemia from VF (Canyon and Dobson, 2004).

Materials and Methods: Male Sprague Dawley rats (300-350 g, fed) are anesthetized with Na-pentabarbitone (60 mg/kg ip) and administered as required throughout the experiment (Ethics approval number A557). The study conforms to the ethical guidelines from the NIH Publication No. 85-23, revised 1996), and by NHMRC. Adenosine, blue dye, triphenyltetrazolium chloride (TTC), adenosine, naloxane and [D-Pen 2, 5] enkephalin (DPDPE) was obtained from Sigma Aldrich (Castle Hill, NSW). Lignocaine hydrochloride was purchased as a 2% solution (ilium). The surgical procedure has been described in Canyon and Dobson (2004). Briefly, a tracheotomy is performed and rats will be ventilated at 75-80 strokes per min using a Harvard Small Animal Ventilator. Body temperature is maintained at 37° C. using a homeothermic blanket control unit. The right and left femoral veins and arteries are cannulated for drug infusions, blood collection and blood pressure monitoring (UFI 1050 BP) using a MacLab. The heart is accessed by a left thoracotomy after removing the 4^{th} and 5^{th} ribs along with the adjoining intercostal muscle. The heart is then gently exteriorized and a 6-0 suture will be quickly threaded under the left coronary artery (LCA) and connected to a reversible snare occluder. Any animal that produces dysrhythmias or a sustained fall in mean arterial blood pressure below 80 mmHg prior to ischaemia is not included. Arrhythmias are analyzed during ischaemia and the first 30 min of reperfusion. Using the lead II ECG tracing, the episodes and duration of episodes of ventricular fibrillation (VF) and ventricular tachycardia (VT) is recorded. VF was defined as a signal where individual QRS deflections could not easily be distinguished from each other and where rate could no longer be measured, and VT was defined as 4 or more consecutive ventricular premature beats. After 120 min reperfusion, the coronary artery was reoccluded and the heart excised. Blue dye (3 ml) was flushed antegrade through the aorta and allowed to circulate through the coronary vasculature to delineate the ischaemic risk zone. The heart was sliced transversely into 6 or 7 slices of uniform thickness (2 mm) and risk area and infarct size estimated as described by Canyon and Dobson (2004). Infarct size is defined as the ratio of the area of necrosis (AN) to the area at risk (AN/AAR) and expressed as a percentage. The primary end-points will be mortality, episodes and duration of ventricular arrhythmias and infarct size. The secondary end-points include heart rate, mean arterial pressure, rate-pressure product (heart rate×systolic pressure) and plasma CK and lactate levels using routine analysis.

Statistical analysis All values are expressed as means±SE of the mean. For infarct size data, a one-way ANOVA is used with a least significance difference (LSD) post hoc test. A Mann-Whitney U test is used for comparison of arrhythmia frequency and duration because the variables of VT and VF are not normally distributed. Haemodynamic data is compared using an ANOVA for repeated measures. Significance is set at a P≦0.05.

1. A method for reducing damage to a cell, tissue or organ following ischemia comprising:
administering an effective amount of a composition including (i) a potassium channel opener or agonist and/or adenosine receptor agonist; and (ii) an antiarrhythmic agent; and
postconditioning the cell, tissue or organ.

2. A method according to claim 1, wherein the composition further includes a delta-1-opioid receptor agonist.

3. A method according to claim 2, wherein the opioid is [D-Pen 2,5]enkaphalin (DPDPE).

4. A method according to claim 1 the adenosine receptor agonist is CCPA.

5. A method for reducing damage to a cell, tissue or organ following trauma comprising:
administering an effective amount of a composition including (i) a potassium channel opener or agonist and/or adenosine receptor agonist; and (ii) an antiarrhythmic agent; and
postconditioning the cell, tissue or organ.

6. A method for reducing damage to a cell, tissue or organ during ischemia or reperfusion:
administering an effective amount of a composition including (i) a potassium channel opener or agonist and/or adenosine receptor agonist; and (ii) an antiarrhythmic agent; and
postconditioning the cell, tissue or organ.

7. A method for reducing damage to a cell, tissue or organ following ischemia comprising administering to the cell, tissue or organ an effective amount of a composition including (i) a potassium channel opener or agonist, and/or adenosine receptor agonist; (ii) an antiarrhythmic agent and (iii) an opioid.

8. A method for reducing damages to a cell, tissue or organ following trauma, comprising administering to the cell, tissue or organ an effective amount of a composition including (i) a potassium channel opener or agonist and/or adenosine receptor agonist; (ii) an antiarrhythmic agent and (iii) an opioid.

9. A method for reducing damage to a cell, tissue or organ during ischemia or reperfusion comprising administering to the cell, tissue or organ an effective amount of a composition including (i) a potassium channel opener or agonist and/or adenosine receptor agonist; (ii) an antiarrhythmic agent and (iii) an opioid.

10. A method according to claim 7, further including postconditioning the cell, tissue or organ.

11. A composition for reducing damage to a cell, tissue or organ during ischemia or reperfusion or following ischemia or trauma comprising:
(i) a potassium channel opener or agonist and/or adenosine receptor agonist;
(ii) an antiarrhythmic agent; and
(iii) an opioid.

12. A method according to claim 8 further including postconditioning the cell, tissue or organ.

13. A method according to claim 9 further including postconditioning the cell, tissue or organ.
